(19) United States
(12) Patent Application Publication
(10) Pub. No.: US 2021/0026449 A1
(43) Pub. Date: Jan. 28, 2021

(54) NEUROFEEDBACK FOCUS AND PRODUCTIVITY SYSTEM, PROCESS, AND METHOD OF USE

(71) Applicants: Siu Man Chan, Hong Kong (CN); Sahin Ozsoy, Baltimore, MD (US)

(72) Inventors: Siu Man Chan, Hong Kong (CN); Sahin Ozsoy, Baltimore, MD (US)

(21) Appl. No.: 16/935,914

(22) Filed: Jul. 22, 2020

Related U.S. Application Data
(60) Provisional application No. 62/877,333, filed on Jul. 23, 2019.
(60) Provisional application No. 62/925,653, filed on Oct. 24, 2019.

(51) Int. Cl. G06F 3/01; A61B 5/0482; A61B 5/00; G06N 20/00

(52) U.S. Cl. CPC G06F 3/015; A61B 5/0482; G06N 20/00; A61B 5/742; A61B 5/6803

(57) ABSTRACT

A neurofeedback focus and productivity system, process, and method of use are presented herein. The neurofeedback focus and productivity system presents a pair of smart earbuds that help people increase their focus and boost their productivity. Additionally, the system can implement various versions and control of various versions of the electronic content, whether the content is used to help a user regain a focus or for another purpose. The electronic content may include storing, processing, and/or replacing data and/or other databases and/or datasets and as a source for creating information from uploaded data.

### CROSS-REFERENCE TO RELATED APPLICATIONS

The present application claims priority to U.S. Provisional Patent Application No. 62/877,333 which was filed on Jul. 23, 2019, and U.S. Provisional Patent Application No. 62/925,653 which was filed on Oct. 24, 2019, which are hereby incorporated by reference herein in their entirety, including any figures, tables, or drawings.

### FIELD OF THE DISCLOSURE

This disclosure relates to a neurofeedback focus and productivity system, process, and method of use. More specifically and without limitation, this disclosure relates to an earpiece system for sensing, collecting data, analyzing data, and software for determining whether a user is distracted or not and providing an audio cue to help train and/or regulate the brain activity, the process of using and related methods of use.

### COPYRIGHT NOTICE

At least a portion of the disclosure of this patent document contains material that is subject to copyright protection. The copyright owner has no objection to the facsimile reproduction by anyone of the patent document or the patent disclosure, as it appears in the Patent and Trademark Office patent files and/or records, but otherwise reserves all copyright rights whatsoever. The following notice applies to the software and data as described below and in the drawings that form a part of this document: Potential X LTD. All rights reserved.

### BACKGROUND OF THE DISCLOSURE

Earphones are old and well known in the art. Conventional earphones have been utilized to remotely play music or other sounds directly into the ears of a user. Earphones (or headphones, or earspeakers, or earphones, or earbuds, or earpieces) traditionally refer to a pair of small loudspeaker drivers worn on or around the head over a user's ears. They are electroacoustic transducers, which convert an electrical signal into a corresponding sound. Earphones allow private listening, by a single user, to an audio source.

Due to the spread of wireless devices in recent years, with the advent of mobile computing devices and music streaming, earphones are increasingly used by people in public places such as sidewalks, grocery stores, airplanes, and in public transit. Earphones are also used by people in various professional contexts, such as audio engineers mixing sound for live concerts or sound recordings and DJs, who use earphones to cue subsequent songs without the audience hearing. Additionally, aircraft pilots and call center employees as well as others who may frequently use earphones in communication. Many who use earphones in communication use an earphone which includes a microphone device. Additionally, people frequently use noise cancelling earphones in public, not for listening to audio, but for cancelling public sound.

Noise cancelling headphones, or noise cancelling earphones are earphones that reduce unwanted ambient noise. Most noise cancelling headphones work by using an active noise control which consists of emitting lower-frequency portions of sound, and incorporation of a microphone that measures ambient sound to generate a waveform that is the exact negative of the ambient sound, and mix the negative waveform generated with any audio signal a listener desires or may be listening to.

Noise cancelling headphones have grown rapidly in popularity. With congested cities, crowded coffee shops, and open-air work environments, unwanted sound from the environment can be reduced by excluding sound from the ear. In this way, earphones can aid the user by assisting a user in focusing. In this way, a person can use noise cancelling headphones technology to block out distractions, so that a person can enjoy music, better to hear sound, and/or better focus on a task at hand.

Said another way, reducing noise distractions is one way concentration can be elevated. A person can get deeper into flow, deeper into their music they are listening to, deeper into work they are trying to complete, tasks they are trying to complete, or whatever else a person wants to focus on. In this way, a person can be exposed to loud environments, neighboring conversations but continue to focus.

Electroencephalography (or “EEG”) is the measuring of brain activity by detecting and measuring brain waves from monitoring and recording electrical activity of the brain. More specifically, EEG measures voltage fluctuations resulting from current within neurons of the brain. EEG, by definition is a measurement of the brain's spontaneous electrical activity, over a period of time. Therefore, measurements of the brain and electrical signals must be measured over a period of time.

Typically, to measure brainwaves, electrodes must be placed at various locations, locations capable of detecting electrical activity taking place within the brain. An electrode is an electrical conductor. In this way, the electrodes placed across a person's scalp detect electrical fluctuations. In this way, EEG is able to detect and measure brainwave activity for a length of time and an analysis can determine whether “stimulus onset” has occurred. Stimulus onset is a change in brain activity from some sort of stimulus, whether visual stimuli, auditory stimulus, and/or somatosensory stimulus. Stimuli are processed in a more complex manner than typical brain thoughts or reactions.

In this way, it is possible to use artificial intelligence and/or machine learning techniques such as neural networks and detection of EEG brainwave data to classify mental states. Some mental states which might desire to be classified are various cognitive states which can influence human behavior such as a relaxed mental state, a neutral mental state, a focused or concentrated mental state, a distracted mental state, and more, including but not limited to other emotional and/or mental states.

Thus, it is a primary object of this disclosure to provide a neurofeedback focus and productivity system, process, and method of use for helping people increase their focus and boost their productivity. In other words, the primary object of this disclosure is to provide a neurofeedback focus and productivity system, process, and method of use for capturing EEG brainwaves using various sensors at optimal locations, analysing and interpreting that data using machine learning and/or artificial intelligence to determine whether a user has become distracted and or needs re-focused, and aiding the user in re-focusing by blocking out the distraction and/or triggering indications to the user that they have become distracted and should refocus.

### SUMMARY OF THE DISCLOSURE

A neurofeedback focus and productivity system, process, and method of use is presented. The neurofeedback focus and productivity system, process, and method of use may include a machine-readable media for electronic management and analysis and for the creation, editing, publication, and distribution of the data gathered. The neurofeedback focus and productivity system, process, and method of use disclosed herein presents an example for the gathering, viewing, editing, and analyzing of EEG data as well as prescribing a treatment and/or action based on the analysis of the data.

In one example, the system disclosed herein is a neurofeedback focus and productivity system, process, and method of use for a pair of smart earbuds that help people increase their focus and boost their productivity. Additionally, the system can implement various versions and control of various versions of the electronic content, whether the content is used to help a user regain a focus or for another purpose. The electronic content may include storing, processing, and/or replacing data and/or other databases and/or datasets and as a source for creating information from uploaded data.

In other words, the system disclosed herein captures brain EEG waves and data using a plurality of sensors located in optimal locations on the scalp and within the ear canal and on the ear. The data gathered from the sensors is then used to determine the mental state of the user. If the mental state of the user is a distracted mental state, then the system causes an action to refocus the user. One way of refocusing a user is by causing an audio cue to play through the speakers of the earbud. Another way of refocusing a user is by increasing the volume of current audio they are listening to. This alternative form can provide a more subtle reminder to the user than other forms which are used and available to the user. The system presented is a small size and can be used in the ear of a human as opposed to an over the scalp sensor and/or other large sensing device as required for commercial EEG measurements and/or other consumer products.

Thus, it is a primary object of the disclosure to provide a neurofeedback focus and productivity system, process, and method of use that increases focus of the user and boosts productivity.

Another object of the disclosure is to provide a neurofeedback focus and productivity system, process, and method of use that captures brain wave data.

Yet another object of the disclosure is to provide a neurofeedback focus and productivity system, process, and method of use that can process brain wave data.

Another object of the disclosure is to provide a neurofeedback focus and productivity system, process, and method of use that has a machine learning capability, able to learn the unique brainwaves and/or brainwave patterns of a user and determine when that particular user has lost focus and/or has entered an altered mental state which is undesired.

Yet another object of the disclosure is to provide a neurofeedback focus and productivity system, process, and method of use that has machine learning capabilities.

Another object of the disclosure is to provide a neurofeedback focus and productivity system, process, and method of use that provides an audio cue to refocus a user.

Yet another object of the disclosure is to provide a neurofeedback focus and productivity system, process, and method of use that can train a brain.

Another object of the disclosure is to provide a neurofeedback focus and productivity system, process, and method of use that can regulate a brain.

Yet another object of the disclosure is to provide a neurofeedback focus and productivity system, process, and method of use that can provide EEG neurofeedback.

Another object of the disclosure is to provide a neurofeedback focus and productivity system, process, and method of use that is small.

Yet another object of the disclosure is to provide a neurofeedback focus and productivity system, process, and method of use that can fit in-ear as opposed to over a scalp.

Another object of the disclosure is to provide a neurofeedback focus and productivity system, process, and method of use that can process EEG brainwave data.

Yet another object of the disclosure is to provide a neurofeedback focus and productivity system, process, and method of use that can store EEG brainwave data.

Another object of the disclosure is to provide a neurofeedback focus and productivity system, process, and method of use that can learn the brainwave data of a user.

Yet another object of the disclosure is to provide a neurofeedback focus and productivity system, process, and method of use that can interpret the brainwave data of any user.

Another object of the disclosure is to provide a neurofeedback focus and productivity system, process, and method of use that can analyze brainwave data.

Yet another object of the disclosure is to provide a neurofeedback focus and productivity system, process, and method of use that offers various versions of refocus techniques including noise cues, musical cues, audio refocusing, noise cancelling, white noise, and more.

Another object of the disclosure is to provide a neurofeedback focus and productivity system, process, and method of use that allows a user to track focus data.

Yet another object of the disclosure is to provide a neurofeedback focus and productivity system, process, and method of use that can determine how different variables affect a user's focus such as, but not limited to, the music a user is listening to, the weather, day of the week, week of the year, temperature, and more.

Another object of the disclosure is to provide a neurofeedback focus and productivity system, process, and method of use that can learn how different variables affect various user's such as the music a user is listening to, the weather, day of the week, week of the year, temperature, location, ambient noise levels, local population, and more.

Yet another object of the disclosure is to provide a neurofeedback focus and productivity system, process, and method of use that allows a user to track historical data.

Another object of the disclosure is to provide a neurofeedback focus and productivity system, process, and method of use that can track the average focus of a user.

Yet another object of the disclosure is to provide a neurofeedback focus and productivity system, process, and method of use that enables a user to monitor brain activity in real time.

Another object of the disclosure is to provide a neurofeedback focus and productivity system, process, and method of use that creates a flow state for a user.

Yet another object of the disclosure is to provide a neurofeedback focus and productivity system, process, and method of use that uses EEG neurofeedback technology.

Another object of the disclosure is to provide a neurofeedback focus and productivity system, process, and method of use that is bluetooth capable.

Yet another object of the disclosure is to provide a neurofeedback focus and productivity system, process, and method of use that has great sound clarity.

Another object of the disclosure is to provide a neurofeedback focus and productivity system, process, and method of use that is accurate.

Yet another object of the disclosure is to provide a neurofeedback focus and productivity system, process, and method of use that provides noise isolation.

Another object of the disclosure is to provide a neurofeedback focus and productivity system, process, and method of use that blocks out the outside world.

Yet another object of the disclosure is to provide a neurofeedback focus and productivity system, process, and method of use that provides for music enjoyment.

Another object of the disclosure is to provide a neurofeedback focus and productivity system, process, and method of use that provides deeper concentration.

Yet another object of the disclosure is to provide a neurofeedback focus and productivity system, process, and method of use that aids a user in sleeping.

Another object of the disclosure is to provide a neurofeedback focus and productivity system, process, and method of use that encourages studying.

Yet another object of the disclosure is to provide a neurofeedback focus and productivity system, process, and method of use that provides for good work habits.

Another object of the disclosure is to provide a neurofeedback focus and productivity system, process, and method of use that provides a pomodoro technique.

Yet another object of the disclosure is to provide a neurofeedback focus and productivity system, process, and method of use that provides operant conditioning.

Another object of the disclosure is to provide a neurofeedback focus and productivity system, process, and method of use that improves the brain's ability to tune out distractions.

Yet another object of the disclosure is to provide a neurofeedback focus and productivity system, process, and method of use that can be used with various digital platforms and various mobile computing devices.

Another object of the disclosure is to provide a neurofeedback focus and productivity system, process, and method of use that provides a user interface.

Another object of the disclosure is to provide a neurofeedback focus and productivity system, process, and method of use that improves upon EEG sensing.

Yet another object of the disclosure is to provide a neurofeedback focus and productivity system, process, and method of use that is easy to use.

Another object of the disclosure is to provide a neurofeedback focus and productivity system, process, and method of use that is quick and efficient.

Yet another object of the disclosure is to provide a neurofeedback focus and productivity system, process, and method of use that can be widely used.

Another object of the disclosure is to provide a neurofeedback focus and productivity system, process, and method of use that can be used in a variety of ways.

Yet another object of the disclosure is to provide a neurofeedback focus and productivity system, process, and method of use that is robust.

Another object of the disclosure is to provide a neurofeedback focus and productivity system, process, and method of use that saves time for a user.

Yet another object of the disclosure is to provide a neurofeedback focus and productivity system, process, and method of use that saves capital for a user.

Another object of the disclosure is to provide a neurofeedback focus and productivity system, process, and method of use that increase productivity for a business.

Yet another object of the disclosure is to provide a neurofeedback focus and productivity system, process, and method of use that is high quality.

These and other objects, features, or advantages of the present disclosure will become apparent from the specification and claims.

### BRIEF DESCRIPTION OF THE DRAWINGS

FIG. 1 is a bottom, perspective view showing the neurofeedback focus and productivity system, process, and method of use; the view showing the main body of the neurofeedback focus and productivity system having a housing; the view showing the neurofeedback focus and productivity system having a loop; the view showing the neurofeedback focus and productivity system having an earbud; the view showing the neurofeedback focus and productivity system having a cable;

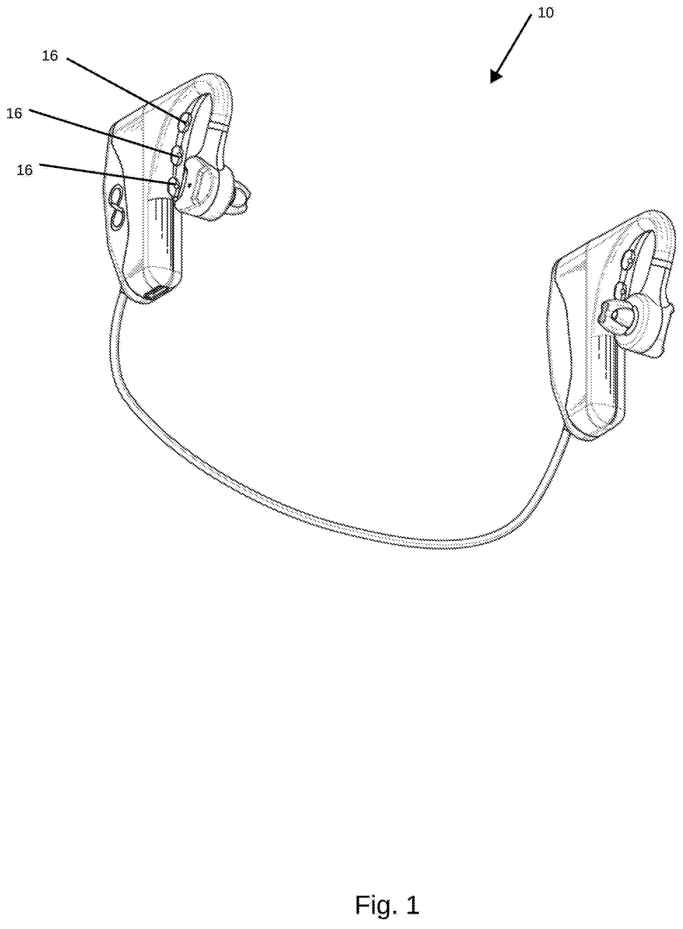

FIG. 2 is a bottom, perspective view showing the neurofeedback focus and productivity system, process, and method of use; the view showing the main body of the neurofeedback focus and productivity system having a housing; the view showing the neurofeedback focus and productivity system having a loop; the view showing the neurofeedback focus and productivity system having an earbud; the view showing the neurofeedback focus and productivity system having a cable;

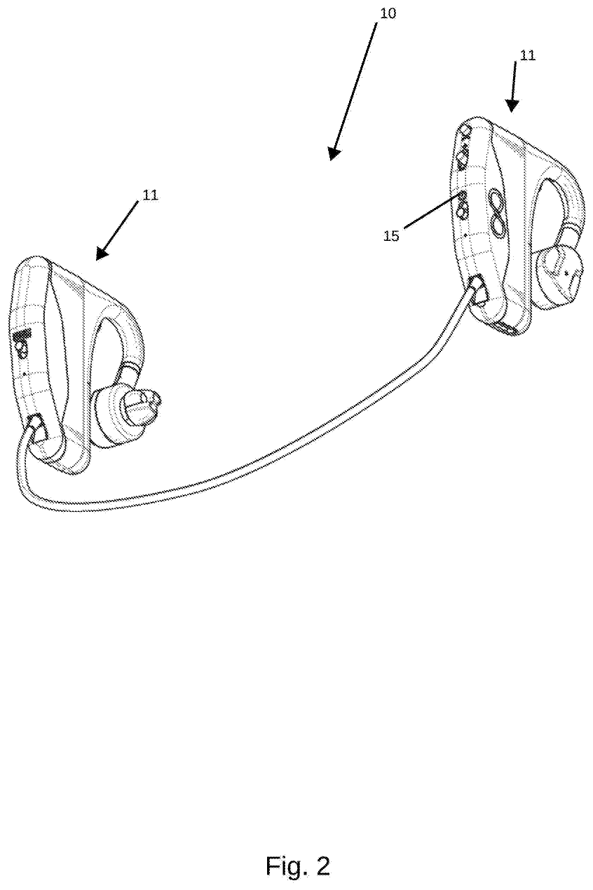

FIG. 3 is a rear, elevation view showing the neurofeedback focus and productivity system, process, and method of use; the view showing the main body of the neurofeedback focus and productivity system having a housing; the view showing the neurofeedback focus and productivity system having a loop; the view showing the neurofeedback focus and productivity system having an earbud; the view showing the neurofeedback focus and productivity system having a cable;

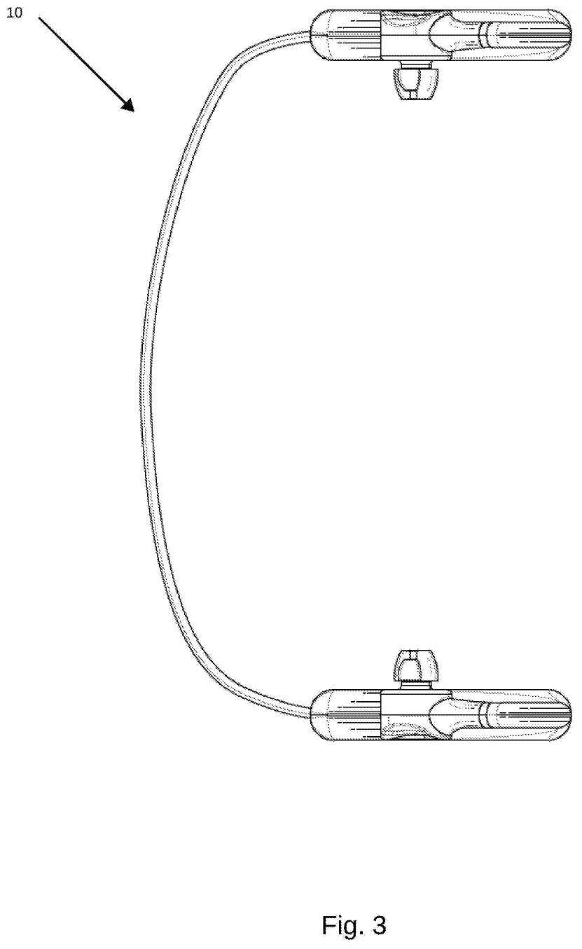

FIG. 4 is a front, elevation view showing the neurofeedback focus and productivity system, process, and method of use; the view showing the main body of the neurofeedback focus and productivity system having a housing; the view showing the neurofeedback focus and productivity system having an earbud; the view showing the neurofeedback focus and productivity system having a cable;

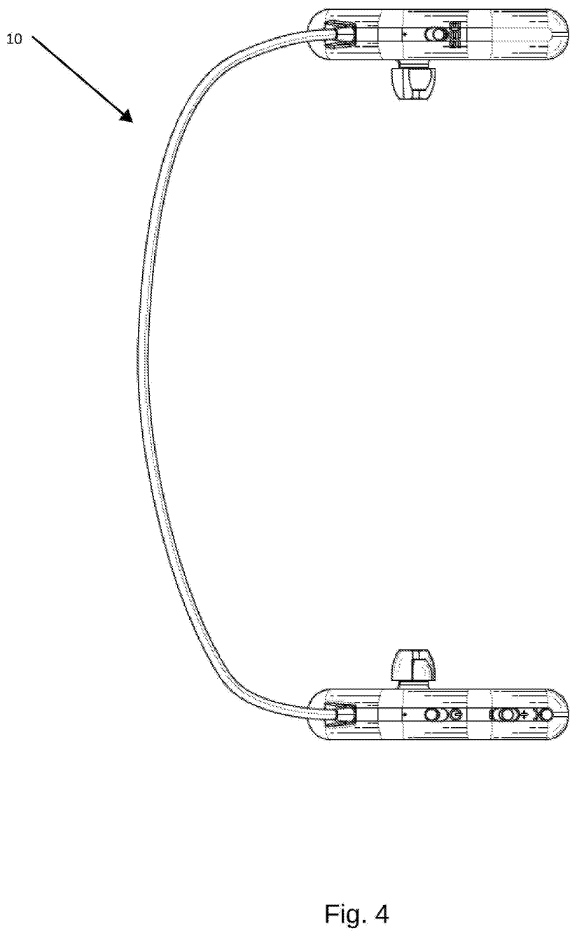

FIG. 5 is a bottom view showing the neurofeedback focus and productivity system, process, and method of use; the view showing the main body of the neurofeedback focus and productivity system having a housing; the view showing the neurofeedback focus and productivity system having a loop; the view showing the neurofeedback focus and productivity system having an earbud; the view showing the neurofeedback focus and productivity system having a cable;

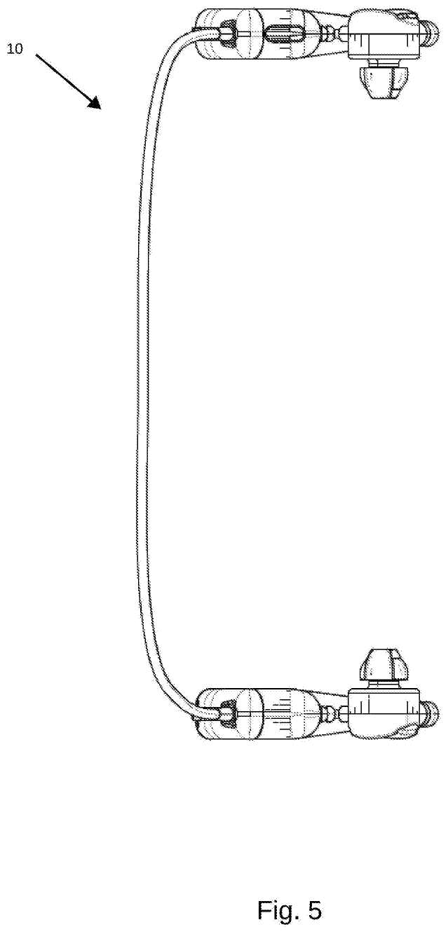

FIG. 6 is a top view showing the neurofeedback focus and productivity system, process, and method of use; the view showing the main body of the neurofeedback focus and productivity system having a housing; the view showing the neurofeedback focus and productivity system having a loop; the view showing the neurofeedback focus and productivity system having an earbud; the view showing the neurofeedback focus and productivity system having a cable;

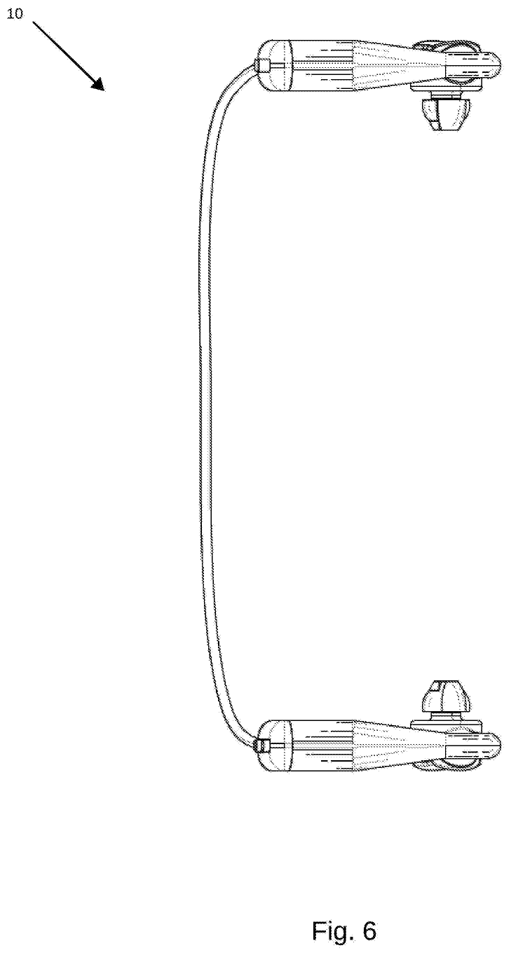

FIG. 7 is a side, elevation view showing the neurofeedback focus and productivity system, process, and method of use; the view showing the main body of the neurofeedback focus and productivity system having a housing; the view showing the neurofeedback focus and productivity system having a loop; the view showing the neurofeedback focus and productivity system having an earbud; the view showing the neurofeedback focus and productivity system having a cable;

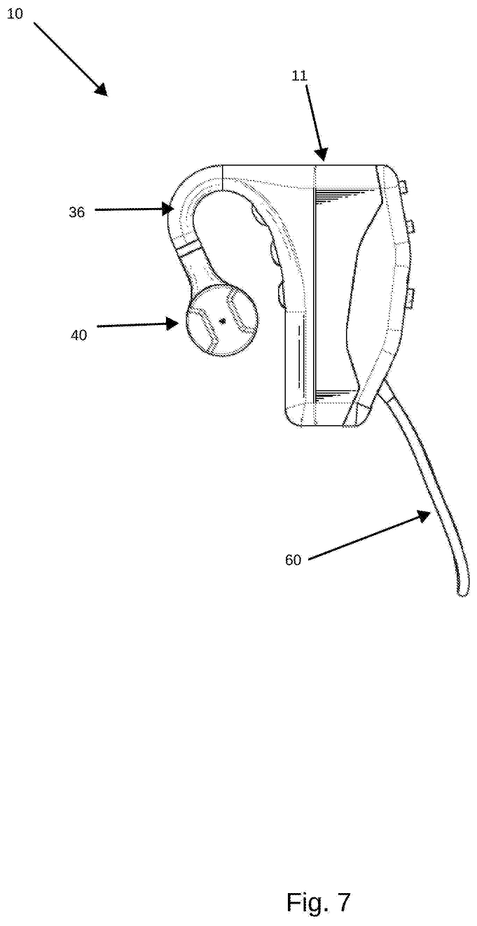

FIG. 8 is a side, elevation view showing the neurofeedback focus and productivity system, process, and method of use; the view showing the main body of the neurofeedback focus and productivity system having a housing; the view showing the neurofeedback focus and productivity system having a loop; the view showing the neurofeedback focus and productivity system having an earbud; the view showing the neurofeedback focus and productivity system having a cable;

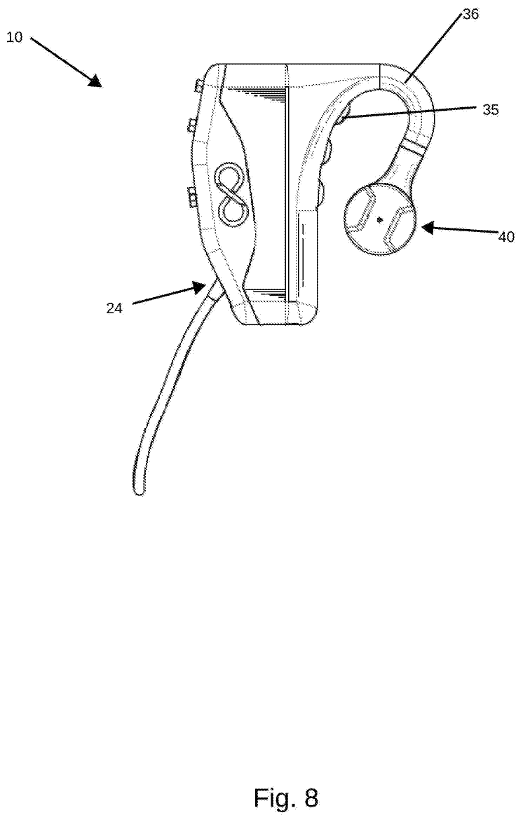

FIG. 9 is an a exploded, isometric view showing the neurofeedback focus and productivity system, process, and method of use; the view showing the main body of the neurofeedback focus and productivity system having a housing; the view showing the neurofeedback focus and productivity system having a loop; the view showing the neurofeedback focus and productivity system having an earbud; the view showing the neurofeedback focus and productivity system having a cable.

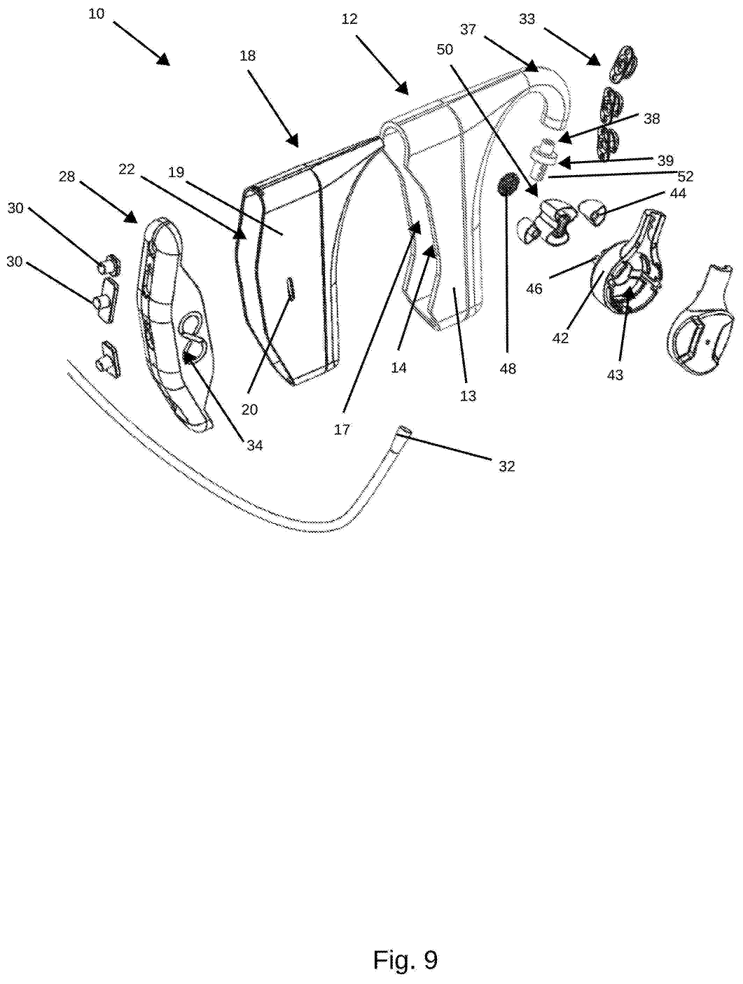

### DETAILED DESCRIPTION OF THE DISCLOSURE

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description, and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium.

Any combination of one or more computer-usable or computer-readable media may be utilized. For example, a computer-readable medium may include one or more of a portable computer diskette, a hard disk, a random access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of the present disclosure may be written in any combination of one or more programming languages. Such code may be compiled from source code to computer-readable assembly language or machine code suitable for the device or computer on which the code will be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“Saas”), Platform as a Service (“PaaS”), and Infrastructure as a Service (“IaaS”)), and deployment models (e.g., private cloud, community cloud, public cloud, and hybrid cloud).

The flowchart and block diagrams in the attached figures illustrate the architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

The neurofeedback focus and productivity system, process, and method of use, disclosed herein, may be formed of any suitable size, shape, and design and is configured to aid a user in focusing and boost productivity. The disclosure herein is configured to integrate with various platforms and/or accept various input from a plurality of sensors which aids in accuracy and productivity. By taking data from multiple inputs and examining that data over a length of time, accuracy is increased and individual and varying behaviors can be accommodated. In this way, the disclosure herein, can aid a user in focusing and increase productivity for any user based on customized data and brainwave behavior of an individual user. Additionally, the disclosure herein, is capable of training a user over the long-term and also providing various cues and training methodologies.

Embodiments of the present disclosure include systems and methods that automatically pull all EEG and other information, such as pulse, body temperature, and other sensed data, into a comprehensive database containing a myriad of information about the individual. This data, combined with age, sex, and other information can be used to create customized information, identify patterns, and more. Furthermore, the disclosure herein, for the first time, allows a user with no psychology and/or behavior analysis knowledge and/or earphone knowledge, to use the system, learn from the system, and be more productive in both a short term and long term setting.

In other words, a user can integrate their contact information, personal information, demographic information, and/or health information and more into thousands of pieces of data to be catalogued and use in data manipulation and analysis to catering to a specific individual without having an intimate knowledge of the individual or medical records from a beginning; additionally without having to have a formal EEG scan done at a hospital or by another specialist, which could costs tens of thousands of dollars.

Furthermore, the disclosure herein allows rapid input of stored information into projects configured in a database as well as rapid computation of the data, as well as rapid response. New users can be added with a minimal amount of configuration. In other words, new users, who have very specific locales or unique personalities, and more, can be integrated into the disclosure herein, the disclosure herein configuring and learning the focus and/or productivity needs of each new user and/or new user. This rapid assimilation of the new user provides for instant use with the disclosure herein, and instant assimilation.

System:

With reference to the figures, a neurofeedback focus and productivity system 10, process, and method of use is presented (hereafter known as “focus system”, “productivity system” or simply “system”). Productivity system 10 is formed of any suitable size, shape and design. In the arrangement shown, as one example, productivity system 10, which may comprise remote servers, databases, application servers, application databases, product databases, mobile applications, and/or computers that fulfill the functions disclosed herein, also includes, in the embodiment(s) depicted, a main body 11, a loop 36, an earband 40, a cable 60, an application server, a processing subsystem, a case, and an onboard computing device, among other components.

Additionally, system 10 may include any suitable configuration, computer processor device, user interface and program and is configured to facilitate the quick, easy, safe, efficient, and accurate processing and/or diagnosing of information interpreted and presented by the performance of behavior analysis.

Main Body:

In the arrangement shown, as one example, system 10 includes a main body 11. Main body 11 is formed of any suitable size, shape, and design and is configured to house and hold a majority of the components of system 10. Additionally, the main body 11 is configured to fit the ear of a user. Main body 11 is shaped in a way that main body 11 fits around the ear of a user, able to engage both the interior and/or ear canal (or external ear canal) as well as engage the pinna and the tympanic membrane.

In the arrangement shown, as one example, main body 11 includes an external housing 12 and an internal housing 18. External housing 12 is formed of any suitable size, shape, and design and is configured to be the outermost portion of the main body 11. In this way the external housing 12 forms the outermost or exterior surface of the main body 11 and provides for most of the appearance of system 10. Additionally exterior housing 12 is configured to attach all of the internally placed components with the loop 36 and the earband 40 which extend as an appendage, in the arrangement shown, from the exterior housing 12 of the main body.

In the arrangement shown, as one example, exterior housing 12 is generally rectangular in shape extending from a top to a bottom having two sidewalls 13 and a hollow interior 17. Additionally, in the arrangement shown, as one example, exterior housing 12 includes snap features 14 and/or connecting features 14. These features connect the exterior housing 12 to the other components of system 10. Additionally, external housing 12 is configured to house and hold indicators 15 as well as EEG sensors 16. In this way, the sensors 16 can be pressed into place when the main body 11 is pressed into place and/or fit onto a user.

In the arrangement shown, as one example, main body 11 includes an internal housing 18. Internal housing 18 is formed of any suitable size, shape, and design and is configured to house and hold the onboard computing device, controls 30, and the various electrical connections of system 10. In the arrangement shown, as one example, internal housing 18 is generally rectangular in shape extending from a top to a bottom having sidewalls 19 and a hollow interior 22. Additionally, in the arrangement shown, as one example, interior housing 18 includes snap features 20 and/or connecting features 20. These features connect the interior housing 18 to the other housing components and features of system 10. Additionally, interior housing 18 is configured to house and hold a power source 24, a printed circuit board (or “PCB”), a power source connector, among other components and/or features.

In the arrangement shown as one example, main body 11 includes a cover 28. Cover 28 is formed of any suitable size, shape, and design and is configured to enclose main body 11. In the arrangement shown, as one example, cover 28 is generally an elongated, hollow, slightly curved shape which is semicircular in shape. In this way, cover 28 is designed to enclose the main body 11 and be ergonomic for housing various controls 30 of system 10. Additionally, cover 28 is configured to house and hold controls 30, cable connector 32, and may hold indicators 33, a display 34, and/or sensors 35, among other components and/or features. Additionally, cover 28 may house a cable connector which may be used to electronically connect one system to another.

In this way, and in the example shown, together, the exterior housing 12, the interior housing 18, the cover 28, and other components and features form the main body 11. In this way, main body 11 is shaped like a helix of the ear with a loop 36 extending toward the ear canal opening.

Loop: In the arrangement shown, as one example, system 10 includes a loop 36. Loop 36 is formed of any suitable size, shape, and design and is configured to fit over the helix and/or structure of the ear of a user in a way that sensors located in optimal position along the exterior housing 12 and possibly the loop 36, itself, are in position for sensing brainwaves and/or detecting brain activity. In other words, and in the arrangement shown, loop 36 is configured to support system 10 in place by supportive and ergonomic shape as well as provide a connection means and a passageway, via a hollow interior 37, to the earbud 40. In the arrangement shown, as one example, loop 36 also includes snap features 38 for connecting to the other components of system 10. Additionally, as one example, in the arrangement shown, a connector 39 is situated as a connecting feature 38 between loop 36 and earbud 40, thereby connecting loop 36 to the earbud 40.

In the arrangement shown, as one example, many of the components of system 10 are connected via a snap feature 14,19,38, however, any connecting means is hereby contemplated for use throughout system 10. Other connecting means for various components of system 10 include, but are not limited to, sealing, bolting, molding, melting, adhering, screwing, snapping, welding, or the like and/or other connection means.

Earbud:

In the arrangement shown, as one example, system 10 includes an earbud 40. Earbud 40 is formed of any suitable size, shape, and design and is configured to provide a housing 42 with a hollow interior 43 which includes, mounted to the exterior, interior, or both, a microphone 44, a speaker 46, a filter 48, at least one sensor 50 and a tip 52, among other components and or features. In the arrangement shown, as one example, earbud 40 is generally circular in shape and designed to protrude, partially into the hollow interior of a portion of the exterior ear canal of a user.

Cable:

In the arrangement shown, as one example, system 10 includes a cable 60. Cable 60 is formed of any suitable size, shape, and design and is configured to connect two systems, or piggy back a second set of sensors and a second earbud in a way that the two connected components work in tandem to cover both ears, sense brain waves, and work simultaneously. In the arrangement shown, as one example, cable 60 provides an electrical connection between the two components. However, a cable 60 is not needed. In an alternative embodiment, system 10 may not have a cable. The two components work in tandem via other forms of communication including, but not limited to, bluetooth and other means of transmitting signals.

Application Server:

In the arrangement shown, as one example, system 10 comprises remote servers, databases, and/or computers that fulfill the functions disclosed and described herein. In the embodiment depicted, system 10 comprises an application server. Application server comprises one or more computer systems adapted to transmit and receive data regarding selected datasets related to various users and/or datasets related to multiple users. Application server is adapted to query database with unique identification codes to retrieve project lists and associated project information related to various users. Application server may transmit project data and/or project documents with respect to a single user and/or multiple users. Application server is also adapted to query a database. This query includes receiving and sending project identification and project data. Additionally, application server may communicate with a mobile application, which can also be adapted to present the data in a form conducive to being viewed on a mobile device and/or handheld device.

The appearance of the presentation of information and/or the process windows for uploading and/or adding and/or sending information to devices and/or databases may vary. As one example, in one embodiment, the appearance of windows and/or the process for refocusing a user, aiding in productivity, and more behavior modification is disclosed herein.

As one of ordinary skill in the art may understand, application server, database, and other databases mentioned herein may be implemented in one or more servers. Furthermore, each may be on multiple servers to increase system efficiency, especially when handling large data gathering, following extended local building codes and/or local building guidelines and/or users. Additionally, multiple servers may have mirrored data to prevent data loss in case of disk failure and/or to decrease access and response times for database queries. In alternative embodiments, application server, and other database procedures may be carried out on computer-readable instructions and data stored on the customer's mobile computing device.

Processing Subsystem:

In the arrangement shown, as one example, system 10 includes a processing subsystem. Processing subsystem is formed of any suitable size shape and design and is configured to allow a user to view interact with, manipulate, and visually access productivity and focus data and information, information related thereto, and/or view various data for various users and/or add information to a profile and/or add information to focus related and/or change the settings of tones, music, and other audio cues and/or change noise cancelling settings and/or other settings.

Processing subsystem is an exemplary method by which systems of the present disclosure may help a user focus, re-focus and/or increase productivity. Employing processing subsystem, enhances a user's interactions with system 10 in the form of awareness and knowledge of data within the system 10. System 10 provides direct links to a user's history, settings, programming, and other information related to behavior and behavior analysis. Additionally, Processing subsystem provides direct interaction with a user's contact information such as phone number, email, and more, as well as vendor website. Additionally, local geographic information with relation to the user can be added, viewed, or retrieved.

Display of Processing subsystem: In the arrangement shown, as one example, system 10 includes a processing subsystem 90. Processing subsystem may include a display, which is configured to show and display information, including data, for review and interpretation by a user. In the arrangement shown, as one example, interactive user display is formed of a display screen, such as that of a desktop computer, laptop computer, monitor, tablet, smart phone, smart TV, projector, virtual reality display or any other device or form of a display. In the arrangement shown, as one example, interactive user display comprises a series of interactive user display pages, however, the interactive user display may consist of a single page or any other method of displaying information on a display as could be adapted to various size screens, devices, or user preferences. The interactive user display can display various patient and/or medical information which is retrieved and/or requested.

Onboard Computing Device:

In one arrangement, as is shown, system 10 includes a computing device connected with electronic network and/or database and/or server or cloud and includes a processor, memory, and may include an interactive user display, among other components. Computing device may be formed of any computing device capable of displaying and manipulating data in the manners described herein. Computing device may include for example a desktop computer, a laptop computer, a tablet, smart phone, or any other computing device or other interactive device.

Computing device may be a single consolidated component, or alternatively, computing device may be formed of a plurality of interconnected components that may be co-located or located at different geographic locations. Computing device may be cloud based or it may be hardware based. In addition, the connected components of computing device, including processor, memory, software and interactive user display, may be co-located with computing device or located at different geographic locations. That is, computing device may be made of any form of a device or system that individually or collectively performs the computing operations of system 10.

Printed Circuit Board: In the arrangement shown, as one example, system 10 includes a printed circuit board (“PCB”). PCB is formed of any suitable size, shape and design and is configured to facilitate carrying and/or holding other components and/or parts necessary to carry out the functions of system 10. PCB, as one example, might be a surface mounted PCB or a through-hole PCB. PCB, as one example, is green and facilitates connecting the components and/or parts of system 10 by the use of traces and or vias. Traces are formed of any suitable size, shape and design and are configured as lines electrically connecting the components and/or parts of system 10. Vias are formed of any suitable size, shape and design and are configured as holes that connect layers of traces together. Generally, as in shown, traces and vias are soldered to connect the components and/or parts to the PCB.

Said another way, onboard computing devices are configured to sense EEG brainwave information and process this information, and cause actions for various interpretations of this information. Some of these actions include, but are not limited to, audio cues, playing music, and noise cancelling. Additionally, onboard computing system is configured to send and receive data once the data is processed. In other words, the onboard computing device is configured to send signals to an offboard computer application and/or offboard system for data processing, storing, and other actions.

In an alternative embodiment, system 10 may not include an onboard computer device, but instead houses only a receiver and/or transceiver for sending and/or receiving information which is both sensed and/or information which is meant to cause the earphones to produce an action. In this way, system 10 may be connected to a mobile computing device via a bluetooth connection, a direction connection, or other connections which provide a means of receiving and/or sending communications.

Microprocessor: Microprocessor is any computing device that receives and processes information and outputs commands according to instructions stored in memory. Memory is any form of information storage such as flash memory, RAM memory, a hard drive, or any other form of memory. Memory may be included as a part of or operably connected to a microprocessor. A receiver/transceiver is connected to a microprocessor. A receiver is used if one way communication is utilized, whereas a transceiver is used if two-way communication is utilized (hereinafter “transceiver”). Receiver/transceiver is connected with an antenna, such as a monopole antenna, a loop antenna, a fractal antenna, or any other form of an antenna. Antenna receives wireless signals from any other device, transmits these signals to receiver/transceiver which processes these signals and then transmits these processed signals to microprocessor which processes these signals according to instructions stored in memory. In one arrangement, system 10 re-transmits operating commands signals through receiver/transceiver so as to similarly control over-the-air communication. Communication is any form of wireless signals, or wired signals, through the air, such as a conventional remote signal, a cell phone, a wireless device, an internet connected device, a hard-wired device, or any other device capable of transmitting remote control signals.

Memory: In the arrangement shown, as on example, system 10 includes a memory. Memory may be formed of any suitable size, shape and design and is configured to facilitate selective storage and retrieval of data (including data) in association with computing devices, processor, software and interactive user display. Memory may be a single component, such as a single chip or drive or other memory device, or alternatively memory may be formed of a plurality of memory or storage components that are connected to one another that may be co-located or located at different geographic locations.

Other Sensors:

In addition to the EEG sensors discussed herein, system 10 may also include other sensors, such as heart rate sensors, temperature sensors, and other sensors. In the arrangement shown, as one example, system 10 includes at least one other sensor 50. Other sensor 50 is formed of any suitable size, shape and design and is configured to facilitate sensing of the outside environment and converting the characteristics of the outside environment into computer readable information. Other sensor 50 is used to detect and respond to some type of input from the physical environment. Other sensor 50 may be used for sensing a single component of an environment, or alternatively another sensor 50 may be used to sense a plurality of components of an environment. For example, the specific input of other sensor 50 may be light, heat, motion, moisture, pressure, or any one of a great number of other environmental phenomena. Other sensor 50 is a device, module, or subsystem whose purpose is to detect events or changes in its environment and send the information to other electronics, frequently a computer processor. The output of another sensor 50 is generally a signal that is generally converted to human-readable display at the sensor location or transmitted electronically over a network for reading or further processing. Both analog sensors and/or digital sensors are hereby contemplated for use. In one arrangement, another sensor 50 and/or microsensor sends information to a processor for use with other electronics.

In addition to the above identified features, options, controls, and components, system 10 may also include a cable 60, a mobile computing device, an application, a case, a case charger, a case dock, among other options, controls, and components.

In Operation:

In the arrangement shown, as one example, focus system 10 is designed to refocus a user when the user loses focus. In this way, productivity of the user is increased. In operation, a user places system 10 over each ear such that the EEG sensors are engaged and/or touching the user. In this way, the EEG sensors are able to detect brain activity. This brain activity and/or brainwave data is collected and processed. Over time, the system 10 recognizes patterns and is able to determine if the wearer has lost focus. If it is determined the wearer has lost focus, via onboard or non-onboard processing software which might include machine learning techniques, then system 10 initiates a predetermined re-focusing action, based either on behavior psychology as an effective means of re-focusing or based on a users preferred method of focusing. This could be playing a tone through a speaker to a user to indicate the user needs to refocus, or initiating noise cancelling functionality, or changing the music that is playing, or begin playing music or a rhythm, or changing the volume of audio. In this operation, system 10 can aid in keeping a user focused, re-focus a distracted user, block out distractions before, during, or after they happen, and overall increase the productivity of a user.

The neurofeedback focus and productivity system, process, and method of use, disclosed herein, is configured to aid a user in focusing and boosting productivity. The disclosure herein is configured to integrate with various platforms and/or accept various input from a plurality of sensors which aids in accuracy and productivity. By taking data from multiple inputs and examining that data over a length of time, accuracy is increased and individual and varying behaviors can be accommodated. In this way, the disclosure herein, can aid a user in focusing and increase productivity for any user based on customized data and brainwave behavior of an individual user. Additionally, the disclosure herein, is capable of training a user over the long-term and also providing various cues and training methodologies.

Embodiments of the present disclosure include systems and methods that automatically pull all EEG and other information, such as pulse, body temperature, and other sensed data, into a comprehensive database containing a myriad of information about the individual. This data, combined with age, sex, and other information can be used to create customized information, identify patterns, and more. Furthermore, the disclosure herein, for the first time, allows a user with no psychology and/or behavior analysis knowledge and/or earphone knowledge, to use the system, learn from the system, and be more productive in both a short term and long term setting.

In other words, a user can integrate their contact information, personal information, demographic information, and/or health information and more into thousands of pieces of data to be catalogued and use in data manipulation and analysis to catering to a specific individual without having an intimate knowledge of the individual or medical records from a beginning; additionally without having to have a formal EEG scan done at a hospital or by another specialist, which could costs tens of thousands of dollars, for a single session or just a few sessions.

Furthermore, the disclosure herein allows rapid input of stored information into projects configured in a database as well as rapid computation of the data, as well as rapid response. New users can be added with a minimal amount of configuration. In other words, new users, who have very specific locales or unique personalities, and more, can be integrated into the disclosure herein, the disclosure herein configuring and learning the focus and/or productivity needs of each new user and/or new user. This rapid assimilation of the new user provides for instant use with the disclosure herein, and instant assimilation.

### Alternative Embodiment

In an alternative embodiment and with reference to the figures, a neurofeedback focus and productivity system, process, and method of use are presented (hereafter known as “focus system”, “productivity system” or simply “system”).

System is formed of any suitable size, shape and design. In the arrangement shown, as one example, a productivity system may comprise an EEG/Audio Head system, and a User Host System such as a mobile device with a mobile application or laptop/desktop computer with a desktop application, an application database, that fulfill the functions disclosed herein. The EEG/Audio Head system and the User Host System has a data link between them, which is denoted as Communication Data. Communication Data may be transferred between and via a wireless communication protocol. Additionally, User Host System may include any suitable configuration, computer processor device, user interface and program and is configured to facilitate the quick, easy, safe, efficient, and accurate processing and/or diagnosing of information interpreted and presented by the performance of behavior analysis.

### Alternative Embodiment—EEG/Audio Head System

EEG/Audio Head System may include, among other components; EEG Sensor Groups, Speaker-1, Speaker-2, and Microphone. EEG/Audio Head System may also include an EEG/Audio Electrical System. EEG Sensor Groups, provide Analog EEG Data to the EEG/Audio Electrical System. Speakers and microphone provide Audio Data to and from the EEG/Audio Electrical System. EEG/Audio Electrical System provides and gets Communication Data to and from the outside world. Communication data may include, but not limited to; Audio data and digital EEG data.

EEG/Audio Electrical System may include, among other components; an Analog Conversion Subsystem, an EEG Data Processing Subsystem, and Audio Data Processing Subsystem. Analog Conversion Subsystem converts the Analog EEG Data to Digital EEG Samples. EEG Data Processing Subsystem processes and generates part of the Communication Data.

Communication Data is composed of Digital Data and Audio Data. Data is used to transmit EEG data to the User Host System and get commands from the User Host System. Audio Data is used to transmit audio data back and forth.

EEG Data Processing Subsystem implements, among other algorithms; an EEG Data Processing Algorithm. The EEG Data Processing Algorithm has, among other functions, filtering and Decimation functions. It filters out the DC component, the mains interference and any frequency components beyond the band of interest. The filtered data then can be decimated to reduce the data rate. The resulting reduced data becomes part of the Digital Data transmitted over the wireless link.

The EEG/Audio Head System can be constructed physically in various ways. One of the possible physical constructions is depicted in FIG. 15. In this depiction, EEG Data Processing Subsystem is housed in an EEG Device, whereas the Audio Data Processing Subsystem is housed in an Audio Device.

In one of the combinations; D1 can be used on the left ear and D2 can be used on the right ear. In one other combination: D1 can be used on the right ear, and D2 can be used on the left ear. There may be a cable connecting D1 and D2.

In the arrangement shown, as one example, EEG/Audio Head system includes D1, D2 and a cable. D1 and D2 are collectively called Devices. Devices are formed of any suitable size, shape, and design and are configured to house and hold a majority of the components. Additionally, Devices are configured to fit the ear of a user. Devices are shaped in a way that they fit around the ear of a user, able to engage both the interior and/or ear canal (or external ear canal) as well as engage the pinna and the tympanic membrane.

EEG Sensor Groups and, as one example, consists of 5 sensors located on D1 and D2 each.

D1 and D2 devices include, in addition to the components described before, in the embodiment(s) depicted, the main body, a Loop, and an Earbud.

In the arrangement shown, as one example, the Main Body includes an External Housing, an Internal Housing and a Cover.

External Housing is formed of any suitable size, shape, and design and is configured to be the outermost portion. In this way the External Housing forms the outermost or exterior surface of the Main Body and provides for most of the appearance of the Earbuds. Additionally, Exterior Housing is configured to attach all of the internally placed components with the Loop and the Earbud which extend as an appendage, in the arrangement shown, from the External Housing.

In the arrangement shown, as one example, is generally rectangular in shape extending from a top to a bottom having two sidewalls and a hollow interior. Additionally, in the arrangement shown, as one example, Exterior Housing includes snap features and/or connecting features to connect it to the other components of the EEG/Audio Head system.

Additionally, is configured to house and hold EEG sensors (M1, M3 and M5 for D1; M2, M4 and M6 for D2). In this way, the EEG sensors can be pressed into place when the main body is pressed into place and/or fit onto a user.

In the arrangement shown, as one example, the main body includes an Internal Housing. Internal housing is formed of any suitable size, shape, and design and is configured to house and hold the EEG/Audio Electrical System, controls and indicators, various electrical connections of EEG/Audio Head System, among other components and/or features. In the arrangement shown, as one example, Internal Housing is generally rectangular in shape extending from a top to a bottom having sidewalls and a hollow interior. Additionally, in the arrangement shown, as one example, Interior Housing includes snap features and/or connecting features. These features connect the Interior Housing to the other housing components and features of the system.

In the arrangement shown as one example, the main body includes a cover. Cover P13 is formed of any suitable size, shape, and design and is configured to enclose the main body. In the arrangement shown, as one example, cover is generally an elongated, hollow, slightly curved shape which is semicircular in shape. In this way, the cover is designed to enclose the main body and be ergonomic for housing various controls of EEG/Audio Head System. Additionally, cover is configured to house and hold controls, and may hold indicators, among other components and/or features. Additionally, cover may house a cable connector which may be used to electronically connect one system to another.

In this way, and in the example shown, together, the External Housing, the Internal Housing, the Cover, and other components and features form the main body. In this way, devices are shaped like a helix of the ear with a Loop and an Earbud extending toward the ear canal opening.

Loop:

In the arrangement shown, as one example, D1 and D2 include a Loop Loop is formed of any suitable size, shape, and design and is configured to fit over the helix and/or structure of the ear of a user in a way that sensors located in optimal position along the External Housing are in position for sensing brainwaves and/or detecting brain activity. In other words, and in the arrangement shown, Loop is configured to support D1 and D2 in place by supportive and ergonomic shape as well as provide a connection means and a passageway, via a hollow, to the Earbud. In the arrangement shown, as one example, Loop also includes snap features for connecting to the other components of D1 or D2.

In the arrangement shown, only one example of connecting the components of D1 and D2 is shown, but other schemes are also possible. Connecting means for various components of system 10 include, but are not limited to, sealing, bolting, molding, melting, adhering, screwing, snapping, welding, or the like and/or other connection means.

Earbud:

In the arrangement shown, as one example, system D1 and D2 include an Earbud. Earbud is formed of any suitable size, shape, and design and is configured to provide a housing with a hollow interior which includes, mounted to the exterior, interior, or both, a microphone, a speaker, a filter, one or two EEG sensors, among other components and or features. In the arrangement shown, as one example, Earbud is generally circular in shape and designed to protrude, partially into the hollow interior of a portion of the exterior ear canal of a user.

Cable:

In the arrangement shown, as one example, EEG/Audio Head System includes a cable. Cable is formed of any suitable size, shape, and design and is configured to connect, in a way that the two connected devices work in tandem to cover both ears, sense brain waves, and work simultaneously. In the arrangement shown, as one example, cable provides an electrical connection between the two components.

However, in general, a cable is not needed. In an alternative embodiment, the EEG/Audio Head System may not have a cable. The two components work in tandem via other forms of communication including, but not limited to, Bluetooth and other means of transmitting signals.

### Alternative Embodiment—Processing Subsystems

In one arrangement, as is shown, EEG/Audio Electrical System includes Processing Subsystems EEG Data Processing Subsystem and Audio Data Processing Subsystem; can be collectively called as “Processing Subsystems”. Processing Subsystems may include microprocessors, DSP chips, FPGAs, memories, among other analog and digital electrical components. They can include one or more computing device(s) capable of manipulating data in the manners described herein. The computing device(s) may include microprocessors, DSP chips, FPGAs or any other computing device. Processing Subsystems may be a single consolidated component, or alternatively, they may be formed of a plurality of interconnected components that may be co-located or located at different ears.

Printed Circuit Board: In the arrangement shown, as one example, EEG/Audio Electrical System includes a printed circuit board, i.e. the components of can be laid out on PCB(s). PCB(s) can be formed of any suitable size, shape and design and is configured to facilitate carrying and/or holding other components and/or parts necessary to carry out the functions. The components on the PCB(s) might be surface mounted or through-hole, or a combination. PCB(s), as one example, can be green and facilitates connecting the components and/or parts by the use of traces and or vias. Traces are formed of any suitable size, shape and design and are configured as lines electrically connecting the components and/or parts. Vias are formed of any suitable size, shape and design and are configured as holes that connect layers of traces together. Generally, traces and vias are soldered to connect the components and/or parts to the PCB(s).

Said another way, EEG/Audio Head System is configured to sense EEG brainwave information and process this information, and cause actions for various interpretations of this information. Some of these actions include, but are not limited to, audio cues, playing music, and noise canceling. Additionally, is configured to send and receive data once the data is processed. In other words, is configured to send signals to an offboard User Host System for data processing, storing, and other actions.

The required processing functions in order for the System to work can be divided between and in various ways. In an alternative embodiment, some processing functions of 11 can be implemented in instead; may be connected to each other via a Bluetooth connection, a direct connection, or other connections that provide a means of receiving and/or sending communications.

As an example, EEG/Audio Head System may include a receiver/transceiver. A receiver is used if one-way communication is utilized, whereas a transceiver is used if two-way communication is utilized (hereinafter “transceiver”). Receiver/transceiver is connected with an antenna, such as a monopole antenna, a loop antenna, a fractal antenna, or any other form of an antenna. The antenna receives wireless signals from any other device, transmits these signals to receiver/transceiver which processes these signals and then transmits these processed signals to Processing Subsystems. In one arrangement, EEG/Audio Head System transmits signals through receiver/transceiver so as to similarly control over-the-air communication. Communication is any form of wireless signals, or wired signals, through the air, such as a cell phone, a wireless device, an internet-connected device, a hard-wired device, or any other device capable of transmitting signals.

Memory: In the arrangement shown, as one example, EEG/Audio Head System (11) includes a memory. The memory may be formed of any suitable size, shape and design and is configured to facilitate selective storage and retrieval of data in association with other parts of the system. The memory may be a single component, such as a single chip or drive or another memory device, or alternatively memory may be formed of a plurality of memory or storage components that are connected to one another that may be co-located or located at different locations.

In addition to the above-identified features, options, controls, and components, system 10 may also include a charging cable, a case, a case charger, and a case dock, among other options, controls, and components.

### Alternative Embodiment—User Host System

User Host System: In the arrangement shown, as one example, System includes a User Host System. User Host System can be, among other possibilities, a mobile device, a laptop computer, or a desktop computer. In fact, any processing device that is capable of establishing a Bluetooth connection can be the User Host System. A Host Application runs on the User Host System. Host Application may use a Data Communication API to send/receive the Digital Data, and an Audio Communication API to send/receive Audio Data. As one example, Host Application may also include, a Machine Learning Component, User Interface, and a Database Component. Data Communication API may also be used by third-party applications to exploit the EEG data.

Host Application may also communicate with a Remote Server. Remote Server may comprise databases, and/or computers that fulfill the functions disclosed and described herein. Remote Server may comprise one or more computer systems adapted to transmit and receive data regarding selected datasets related to various users and/or datasets related to multiple users. It can be adapted to query a database with unique identification codes to retrieve population data and associated feature data related to various users. It may transmit project data and/or project documents with respect to a single user and/or multiple users. It is also adapted to query a database. This query includes receiving and sending project identification and project data. Additionally, Remote Server may communicate with the Host Application, which can also be adapted to present the data in a form conducive to being viewed on a desktop computer, laptop, mobile device and/or handheld device.

The appearance of the presentation of information and/or the process windows for uploading and/or adding and/or sending information to the device and/or databases may vary. As one example, in one embodiment, the appearance of windows and/or the process for refocusing a user, aiding in productivity, and more behavior modification is disclosed herein.

As one of ordinary skill in the art may understand, Remote Server mentioned herein may be implemented in one or more servers. Furthermore, each may be on multiple servers to increase system efficiency, especially when handling large data gathering, following extended local building codes and/or local building guidelines and/or users. Additionally, multiple servers may have mirrored data to prevent data loss in case of disk failure and/or to decrease access and response times for database queries.

User Interface:

In the arrangement shown, as one example, Host Application includes a User Interface. User Interface is formed of any suitable size shape and design and is configured to allow a user to view interact with, manipulate, and visually access productivity and focus data and information, information related thereto, and/or view various data for various users and/or add information to a profile and/or add information to focus related and/or change the settings of tones, music, and other audio cues and/or change noise-canceling settings and/or other settings.

User Interface is an exemplary method by which systems of the present disclosure may help a user focus, re-focus and/or increase productivity. Employing the User Interface enhances a user's interactions with System in the form of awareness and knowledge of data within the System. System provides direct links to a user's history, settings, programming, and other information related to behavior and behavior analysis. Additionally, User Interface provides direct interaction with a user's contact information such as phone number, email, and more, as well as vendor website. Additionally, local geographic information with relation to the user can be added, viewed, or retrieved.

Display of Graphical User Interface: In the arrangement shown, as one example, User Interface may include a display, which is configured to show and display information, including data, for review and interpretation by a user. In the arrangement shown, as one example, interactive user display is formed of a display screen, such as that of a desktop computer, laptop computer, monitor, tablet, smartphone, smart TV, projector, virtual reality display or any other device or form of a display. In the arrangement shown, as one example, User Interface comprises a single or a series of interactive user display page(s). The interactive user display page(s) may employ displaying information as could be adapted to various size screens, devices, or user preferences. The interactive user page(s) can display various user information which is retrieved and/or requested.

Machine Learning Component

In the arrangement shown, as one example, Host Application includes a Machine Learning Component. Machine Learning Component receives Multi-Channel EEG Data and generates Mental State Metrics. Mental State Metrics include, but are not limited to, Focus metrics. In order to generate these metrics, can use, among other algorithms, Epoch Generation, Artifact Detection, Feature Extraction and Classification.

Classifiers can use supervised or unsupervised algorithms. In the example shown in FIG. 12, a supervised algorithm uses population features and the user's own features to generate the Mental State Metrics.

### Alternative Embodiment—Operation of the System

In the arrangement shown, as one example, System is designed to refocus a user when the user loses focus. In this way, the productivity of the user is increased. In operation, a user places EEG/Audio Head System over each ear such that the EEG sensors are engaged and/or touching the user. In this way, the EEG sensors are able to detect brain activity. This brain activity and/or brainwave data is collected and processed. Over time, the System recognizes patterns and is able to determine if the wearer has lost focus. If it is determined the wearer has lost focus, via Machine Learning Component of the Host Application, then Host Application initiates a predetermined re-focusing action, based either on behavior psychology as an effective means of re-focusing or based on a user's preferred method of focusing. This could be playing a tone through a speaker to a user to indicate the user needs to refocus, or initiating noise-canceling functionality, or changing the music that is playing, or begin playing music or a rhythm, or changing the volume of audio. In this operation, System can aid in keeping a user focused, re-focus a distracted user, block out distractions before, during, or after they happen, and overall increase the productivity of a user.

The neurofeedback focus and productivity system, process, and method of use, disclosed herein, is configured to aid a user in focusing and boosting productivity. The disclosure herein is configured to integrate with various platforms and/or accept various input from a plurality of sensors which aids in accuracy and productivity. By taking data from multiple inputs and examining that data over a length of time, accuracy is increased and individual and varying behaviors can be accommodated. In this way, the disclosure herein, can aid a user in focusing and increase productivity for any user based on customized data and brainwave behavior of an individual user. Additionally, the disclosure herein, is capable of training a user over the long-term and also providing various cues and training methodologies.

Embodiments of the present disclosure include systems and methods that automatically pull all EEG and other information, into a comprehensive database containing a myriad of information about the individual. This data, combined with age, sex, and other information can be used to create customized information, identify patterns, and more. Furthermore, the disclosure herein, for the first time, allows a user with no psychology and/or behavior analysis knowledge and/or earphone knowledge, to use the system, learn from the system, and be more productive in both a short term and long-term setting.

In other words, a user can integrate their contact information, personal information, demographic information, and/or health information and more into thousands of pieces of data to be catalogued and use in data manipulation and analysis to catering to a specific individual without having an intimate knowledge of the individual or medical records from a beginning; additionally without having to have a formal EEG scan done at a hospital or by another specialist, which could costs tens of thousands of dollars, for a single session or just a few sessions.

Furthermore, the disclosure herein allows rapid input of stored information into projects configured in a database as well as rapid computation of the data, as well as rapid response. New users can be added with a minimal amount of configuration. In other words, new users, who have very specific locales or unique personalities, and more, can be integrated into the disclosure herein, the disclosure herein configuring and learning the focus and/or productivity needs of each new user and/or new user. This rapid assimilation of the new user provides for instant use with the disclosure herein, and instant assimilation.

It will be appreciated by those skilled in the art that other various modifications could be made to the system, process, and method of use without parting from the spirit and scope of this disclosure. All such modifications and changes fall within the scope of the claims and are intended to be covered thereby.

What is claimed:

1. A neurofeedback focus and productivity system, comprising:
a database;
a main body;
the main body having an external housing;
the main body having an internal housing;
the main body having a cover;
a plurality of sensors;
wherein the plurality of sensors detect brain activity;
an earbud;
the earbud having a speaker;
the earbud having a microphone;
a processor;
wherein the processor interprets brain activity data;
wherein a refocus activity is performed by the earbud when triggered by the processor.

2. The system of claim 1, further comprising:
wherein the refocus activity is performed by the earbud when a set of rules are met based on a predetermined set of instructions.

3. The system of claim 1, further comprising:
a cable; and
an onboard computing system.

4. The system of claim 1, further comprising:
an application server.

5. The system of claim 1, further comprising:
the main body further comprising a plurality of snap features;
wherein the snap features hold the structure of the main body together.

6. The system of claim 1, further comprising:
an interactive user display.

7. The system of claim 1, further comprising:
a power source.

8. The system of claim 1, further comprising:
a power source;
wherein the power source is a plurality of batteries.

9. The system of claim 1, further comprising:
a case system;
wherein the case system charges power to the neurofeedback and productivity system when the neurofeedback and productivity system is engaged within the case.

10. The system of claim 1, further comprising:
an antenna;
a microprocessor;
a memory.

11. A neurofeedback focus and productivity system, comprising:
an EEG audio head system;
a plurality of sensors;
wherein the plurality of sensors detects brain activity;
at least one speaker;
at least one microphone;
an EEG audio electrical system;
the EEG electrical system having an analog conversion subsystem;
the EEG electrical system having at least one processing subsystem;
wherein EEG audio head system comprises a EEG device and an audio device;
the EEG device having a main body;
the EEG device having a loop;
the EEG device having at least one earbud;
the main body of the EEG device having an internal housing;
the main body of the EEG device having an external housing;
the main body of the EEG device having a cover;
a user host system;
the user host system having a plurality of communication application programming interfaces;
the user host system having a user interface.

12. The system of claim 11, further comprising:
the user host system having at least one remote server.

13. The system of claim 11, further comprising:
the user host system having a plurality of databases.

14. The system of claim 11, further comprising:
the user host application having a machine learning feature;
the machine learning feature having a predetermined set of rules;
wherein the machine learning feature makes changes to the predetermined set of rules based on a new set of parameters.

15. The system of claim 11, further comprising:
the user host system having an interactive user display.

16. The system of claim 11, further comprising:
a cable.

17. The system of claim 11, further comprising:
a case.

18. A neurofeedback focus and productivity process, the process comprising:
providing an application server;
providing a product database, the product database having a dataset having data;
providing a plurality of EEG sensors;
providing a processor with machine learning;
interpreting data to learn habits of a user;
recognizing a loss of focus from brainwave data analysis;
sending a refocus signal to refocus a user and enhance productivity.

19. The process of claim 18, further comprising the steps:
providing a user host system;
detecting environmental parameters through a plurality of sensors;
processing information related to the environmental parameters.

20. The process of claim 18 further comprising the steps:
providing a user host system;
detecting environmental parameters through a plurality of sensors;
processing information related to the environmental parameters;
providing a machine learning feature;
providing a predetermined set of rules for the functionality of the user host system;
detecting a new set of environmental parameters after a frequency of time;
changing the predetermined set of rules based on the new set of environmental parameters.
